(19) United States
(12) Patent Application Publication
(10) Pub. No.: US 2008/0300525 A1
(43) Pub. Date: Dec. 4, 2008

(54) Ankle Foot Orthosis Device

(75) Inventor: Tal Shlomovitz, Givataim (IL)

Correspondence Address: PATWRITE LLC, 408 W. MAIN ST., MARSHALLTOWN, IA, 50158-5759, US

(73) Assignee: YAAD ADVANCED ORTHOPEDICS LTD, Tel Aviv (IL)

(21) Appl. No.: 11/757,697

(22) Filed: Jun. 4, 2007

(51) Int. Cl. A61F 5/00

(52) U.S. Cl. 602/28

(57) ABSTRACT

The present invention discloses an ankle foot orthosis device that is adjustable to a patient's drop foot. The device may include, inter alia, a foot-support that is sized to fit into a corresponding patient's footwear; an elongate member that is mechanically coupled to the foot-support; and a lower leg holder that is coupled to the elongate member. The lower portion of the elongate member may extend from the foot-support's heel portion upwards and rearwards towards a corresponding lower leg's calf and may further extend forwardly towards the corresponding lower leg's shin. Forward tilting of the lower leg in the gait cycle causes the elongate member to bend forward, which in turn causes the development of potential energy in the device that may be released during the gait cycle's toe-off and swing phase, thereby compensating for the energy lost in the drop foot.

### FIELD OF INVENTION

This invention relates generally to orthotic devices. More specifically, the present invention relates to orthotic devices that assist people suffering from drop foot condition.

### BACKGROUND OF THE INVENTION

Glossary
a) “ankle”—the joint between the lower leg and the foot
b) “calf”—posterior portion of the lower leg
c) “drop foot condition”—medical condition describing a patient's inability in pivoting a foot upward
d) “drop foot”—the patient's foot suffering from the drop foot condition
e) “extensors”—muscle that straightens a body part
f) “foot”—the lower extremity of the lower leg below the ankle
g) “heel bone”—the largest tarsal bone
h) “knee”—joint in the middle of the leg
i) “lower leg”—part of the leg between the knee and the foot
j) “shin”—anterior part of the lower leg

Drop foot condition, also known as steppage gait, is a medical condition that pertains to a deficit in pivoting the foot upward, which may be of neurological and/or muscular and/or anatomical in origin. For example, an individual may have a weakness in his/her dorsiflexor muscle, which is the muscle that helps the body control and lift the foot during different phases of walking. Because the patient suffering from drop foot condition lacks full control of his foot, difficulties arise when walking such as dragging the toe when swinging the leg forward or slapping the foot when the heel strikes the ground. Any number of medical conditions including nerve damage, stroke, cerebral palsy, and diabetes can cause the drop foot condition. Treatments available include surgery and nerve stimulation. The simplest and certainly least invasive treatment is the use of an ankle foot orthosis, or AFO.

Knee crouch, a condition sometimes associated with drop foot condition, may also be helped by the use of an ankle foot orthosis. Knee crouch is the over-flexion of the knee during the mid-stance phase, causing the knee to collapse forward. This pathology may be caused by, for example, weak knee extensors and/or neurological impairment such as femoral nerve damage. If left uncorrected, knee crouch can lead to early joint degeneration.

Ankle foot orthoses are intended to provide stability and maintain the foot in the proper position during various phases of the walking cycle.

U.S. Pat. No. 3,916,886, which is incorporated by reference in its entirety herein, discloses a preformed, light weight, drop foot brace that is self-conforming and removably positionable adjacent to the posterior calf portion, the heel and instep of a leg of a user who has suffered a stroke or other disability, with the brace when employed maintaining the foot of a user in a normal walking position relative to the leg. The brace is of such structure that both the foot of the user and lower portion of the brace may be disposed in a shoe without alteration or modification of the latter. Due to the conforming nature of the brace it is inconspicuous when worn, for the upper portion of the brace is situated within the confines of the trouser leg of a user, and the lower portion of the brace within a shoe.

Patent Publication No. WO0135876, which is incorporated by reference in its entirety herein, discloses a device which is characterized in that the joint to the foot of a spring attached to the front side of a wearer's leg includes an engagement piece attached in the instep area of the foot in which easy sliding of the spring in the longitudinal direction thereof is allowed. The device allows easy angular bending of the foot against the force of the spring during walking. The foot is also easily returning back to the resting position thereof at an essentially right angle in relation to the leg. Abrasion of the foot, instep or ankle is essentially prevented. Footwear is easy to provide for use of the device of the invention without any uncomfortable accessories inside the footwear.

U.S. Pat. No. 4,672,955, which is incorporated by reference in its entirety herein, discloses thigh and calf bands of a knee orthosis, wherein the thigh and calf bands are formed of a layer (or layers or curable composite material. The sidebars are integral with the thigh and calf bands in that they are simply extensions of the layers forming the thigh and calf bands. The sidebars are cured prior to fitting the thigh and calf bands about the leg, but the thigh and calf bands are left incured so that they may be formable about the leg and cured in place thereon. A method for fabricating the orthosis is disclosed and a kit therefor is described. The invention is also suitable for an ankle orthosis.

U.S. Pat. No. 5,897,515, which is incorporated by reference in its entirety herein, discloses an ankle-foot orthosis made of a carbon fiber reinforced material having low weight that is carried on the front of the lower leg, extending over the lateral ankle and preventing plantar flexion. The orthosis may be worn under ordinary clothes and shoes and promotes a more natural gait pattern. The ankle-foot orthosis comprises a frame of thin flexible material extending over the front of the lower leg, anterior of the lateral ankle and beneath the sole of the foot and a supporting portion of rigid material extending over a narrow part of the front of the lower leg, anterior of the lateral ankle and beneath the part of the sole of the foot. The orthosis also comprises a fastening means for fastening the orthosis to the leg. In a preferred embodiment the orthosis comprises a substantially inflexible reinforcement element and a tough flexible element, the reinforcement element extending over a narrow part of substantially the whole frame and the flexible part extending over a substantial part of the sole of the foot. The frame is preferably made of thin flexible fiber glass reinforced plastic resin material, said reinforcement element being made of rigid carbon fiber reinforced plastic resin material. Said tough flexible element is preferably made of aramid fiber reinforced plastic resin.

U.S. Pat. No. 6,102,881, which is incorporated by reference in its entirety herein, discloses a drop foot brace that includes an upper support bearing against the rear lower leg and a lower support bearing against the rear heel. A springed hinge couples the upper and lower supports and biases the upper and lower supports against the rear of the user's leg and rear of the user's heel, respectively. A shoe maintains the springed hinge against the rear of the user's leg in the vicinity of the user's ankle whereby the user experiences a lifting force in opposition to the foot. The brace needs no coupling to the body and includes sufficient flexibility to facilitate comfort when not in use while still providing aid against foot drop. One form of the foot brace includes the heel or foot-support integrally formed within a shoe and including a slot formation receiving the remaining portions of the brace whereby the brace may be easily removed from the shoe when not needed.

U.S. Pat. No. 6,361,517, which is incorporated by reference in its entirety herein, discloses a foot lift assist that enables a person with foot drop to walk in a nearly normal manner. The foot lift assist comprises an elastic cord anchored at a person's hip by a belt. A foot strap is worn on the foot generally over the toes of the foot. The elastic cord is connected to a ring on the foot strap that is located to the outside of the longitudinal centerline of the foot. The elastic cord then lies completely to the outside of the leg and knee. When the person removes his weight from the foot, the elastic cord exerts an upward force that bends the leg at the knee and hip and that also pivots the foot upwardly about the ankle. The person can then take a step without his toes dragging on the ground. The foot strap may be fully flexible. Alternately, for walking on abrasive surfaces, the foot strap may have a metal plate bonded under a strap. A heel band attached to the plate and looped around the heel helps retain the foot strap on the foot. However, with no lateral stability, the foot may still slap the ground.

U.S. Pat. No. 6,790,193, which is incorporated by reference in its entirety herein, discloses a foot lifter orthosis including a dorsal leg shell, extending over the rear foot, which can be fixed by a strapping means to the calf of the orthosis wearer and which has a forefoot shell section constructed in one piece which extends to a point below the foot sole region, wherein the leg shell exhibits a low resistance in dorsal foot bending and a high resistance compensating for the foot and shoe mass in plantar foot bending with at least one resilient inlay, which extends approximately parallel to and at a short distance from an edge of the leg shell from the calf region thereof to a point below the forefoot shell section. An associated method of formation is also disclosed.

However, the above-referenced devices may be cumbersome to use and may not be useful in helping clear the foot from the floor, since they are inefficient in compensating the energy loss caused in foot suffering from drop foot condition. Furthermore, the devices are not adjustable to the different needs of different patients.

### SUMMARY OF THE INVENTION

The present invention discloses an ankle foot orthosis device adjustable to patient's drop foot for assisting the patient in completing substantially regularly a gait cycle,

In embodiments of the invention, the device may include a foot-support that is sized to fit into corresponding footwear of the patient's drop foot.

In embodiments of the invention, the device may include an elongate member that is mechanically coupled to the foot-support. The elongate member may have a lower portion prolonging to an upper portion. The device may further include a lower-leg-holder that is mechanically coupled to the elongate member. The lower-leg-holder may include a support-brace that enables a corresponding shin to rest thereon.

In embodiments of the invention, the lower portion of the elongate member may extend from a heel portion of the foot-support upwards and rearwards towards a corresponding lower leg's calf, wherein the upper portion may further extend forwardly in a direction corresponding to the lower leg's shin.

In embodiments of the invention, forward tilting of the lower leg against the support-brace causes the elongate member to bend forward in a direction corresponding to the patient's gait, wherein the forward bending causes the development of potential energy in the ankle foot orthosis device. At least some of the potential energy may be released during the toe-off phase of the gait cycle, thereby at least partially compensating for the energy lost due to the drop foot condition.

In embodiments of the invention, some of the potential energy may be released during gait phases subsequent to the toe-off phase.

In embodiments of the invention, the elongate member of the ankle foot orthosis device may be flexible.

In embodiments of the invention, the foot-support of the ankle foot orthosis device is flexible.

In embodiments of the invention, the foot-support may approximately weigh, for example, 70 grams, 60 grams, 50 grams, 40 grams or 30 grams.

In embodiments of the invention, the elongate member may approximately weigh, for example, 100 grams, 90 grams, 80 grams, 70 grams or 60 grams.

In embodiments of the invention, lower leg holder may approximately weigh, for example, 20 grams, 25 grams, 30 grams, 35 grams, 40 grams, 45 grams or 50 grams.

In embodiments of the invention, the device weighs approximately, for example, 500 grams, 400 grams, 350 grams and the like.

In embodiments of the invention, the foot-support may include at least one of the following first materials: carbon fibers, and graphite fibers.

In embodiments of the invention, the first material may be disposed within at least one of the following second materials: epoxy resin, polydicyclopentadiene, and polyimide.

In embodiments of the invention, the density and the structure of the first material and/or the second material may dictate the mechanical properties of the foot-support.

In embodiments of the invention, the foot-support may be ergonomically designed to substantially fit the natural contour of a sole of the drop foot of the patient.

In embodiments of the invention, the foot-support may be sized to fit easily, snugly and securely inside the patient's corresponding footwear.

In embodiments of the invention, the mechanical properties of the foot-support may be engineered as to enable substantial fitting of the foot-support with the natural contour of a sole of a patient's drop foot during all phases of the patient's gait cycle.

In embodiments of the invention, the elongate member may be integrally formed with the foot-support.

In embodiments of the invention, the elongate member may be fixedly coupled to the foot-support with at least one elongate-member-fastener.

In embodiments of the invention, the support-brace may be secured to some portion of the shin by straps.

In embodiments of the invention, the elongate member may be made out of at least one of the following materials: high carbon steel, and very high carbon steel. However, the elongate member retains spring characteristics, i.e., elongate member is flexible.

In embodiments of the invention at least some parts of the elongate member may be shaped and fixedly coupled to foot-support via the at least one elongate-member-fastener such that elongate member is substantially aligned with the anatomic axis of ankle of the patient's drop foot.

In embodiments of the invention, the lower portion of the elongate member extends from a heel portion substantially in alignment with the drop foot's ankle in a direction corresponding to a posterior position with regard to the drop foot's heel bone and ankle, wherein the posterior position may be substantially above the heel bone and the ankle.

In embodiments of the invention, the at least one elongate-member-fastener and/or the elongate member and/or the foot-support are replaceable.

In embodiments of the invention, the at least one elongate-member-fastener includes a connector comprising of a base that may be integrally formed with a protruding plate.

In embodiments of the invention, the protruding plate may be positioned adjacent to the inner side of drop foot, such to enable the fixedly coupling of the elongate member to the protruding plate by fastener means.

In embodiments of the invention, padding elements may be fitted onto the foot-support to provide comfortable cushioning for the drop foot's sole.

The present invention further discloses a method of fabricating an ankle foot orthosis device for assisting a patient having a drop foot.

In embodiments of the invention, the method may include, for example, the act of fabricating a foot support having a heel portion by, for example, laying up several layers of composite material.

In embodiments of the invention, the method may include, for example, the act of fabricating an elongate member that may have a lower and an upper portion that are bent relative to each other and wherein the upper portion is a prolongation of the lower portion.

In embodiments of the invention, the method may include, for example, the act of fabricating a lower-leg-holder, which may include a U-shaped support-brace having an inner surface.

In embodiments of the invention, the method may include, for example, the act of mechanically coupling the elongate member to a heel portion of the foot-support such that the lower portion extends rearwardly from the heel portion towards the drop foot's corresponding calf and such that the upper portion extends anteriorly towards the drop foot's corresponding shin.

In embodiments of the invention, the method may include, for example, the act of mechanically coupling the lower-leg-holder to the elongate member such that a curved part of the support-brace substantially points in a direction that corresponds to the patient's direction of gait, thereby enabling the patient to rest his/her lower leg that corresponds to the drop foot against the inner surface of the support-brace.

### BRIEF DESCRIPTION OF THE DRAWINGS

The subject matter regarded as the invention will become more clearly understood in light of the ensuing description of embodiments thereof, given by way of example only, with reference to the accompanying figures (FIGs.), wherein:

FIG. 1 is a schematic isometric assembly view of an ankle foot orthosis device, according to some embodiments of the invention;

FIG. 2 is a schematic isometric exploded view of the device's elongate member and lower-leg-holder, according to some embodiments of the invention;

FIG. 3 is a schematic side view of the ankle foot orthosis device adjusted to a patient's drop foot, according to some embodiments of the invention;

FIG. 4A is a schematic isometric illustration of a foot-support, according to some embodiments of the invention;

FIG. 4B is a schematic cross-sectional side view of the foot-support, according to some embodiments of the invention;

FIG. 5 is a schematic illustration of the initial-contact phase of a gait cycle performed by the patient's drop foot having adjusted thereon the ankle foot orthosis device, according to some embodiments of the invention;

FIG. 6 is a schematic illustration of the mid-stance phase of the gait cycle performed by the patient's drop foot having adjusted thereon the ankle foot orthosis device, according to some embodiments of the invention;

FIG. 7 is a schematic illustration of the terminal-stance phase of the gait cycle performed by the patient's drop foot having adjusted thereon the ankle foot orthosis device, according to some embodiments of the invention;

FIG. 8 is a schematic illustration of the toe-off phase of the gait cycle performed by the patient's drop foot having adjusted thereon the ankle foot orthosis device, according to some embodiments of the invention;

FIG. 9 is a schematic exploded view of the device's foot-support, the lower-leg-holder, and the corresponding elongate-member-fastener(s), according to some embodiments of the invention; and

FIG. 10 is schematic flow-chart illustration of a method for fabricating the ankle foot orthosis device, according to some embodiments of the invention.

The drawings taken with description make apparent to those skilled in the art how the invention may be embodied in practice.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate identical elements.

### DESCRIPTION OF THE INVENTION

The present invention relates to an ankle foot orthosis (AFO) device. According to some embodiments of the invention, the AFO device includes a flexible foot-support to which a flexible elongate member is substantially fixedly coupled. The AFO device further includes a lower-leg-holder that is mechanically coupled to an upper part of the elongate member. The AFO device is designed in a manner such that when it is suitably engaged with a patient's drop foot, the AFO device stores potential energy during, for example, the transition from the mid-stance to the terminal-stance phase of the patient's gait cycle. At least some of the stored potential energy may be released during the subsequent toe-off phase of the patient's gait cycle, whereby the released potential energy may at least partially compensate for, e.g., the muscle weakness in the drop foot. Consequently, the AFO device may cause ground clearance of the patient's drop foot during at least some of the gait cycle's swing phases.

An embodiment is an example or implementation of the invention. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions.

It is understood that the phraseology and terminology employed herein is not to be construed as limiting, and is for descriptive purpose only.

The principles and uses of the teachings of the present invention may be better understood with reference to the accompanying description, figures and examples.

It should be understood that the details set forth herein do not construe a limitation to an application of the invention. Furthermore, it should be understood that the invention can be carried out or practiced in various ways and that the invention can be implemented in embodiments other than the ones outlined in the description below.

It should be understood that the terms “including”, “comprising”, “consisting” and grammatical variants thereof do not preclude the addition of one or more components, features, steps, integers or groups thereof and that the terms are not to be construed as specifying components, features, steps or integers.

If the specification or claims refer to “an additional” element, that does not preclude there being more than one of the additional element.

It should be understood that where the claims or specification refer to “a” or “an” element, such reference is not to be construed as there being only one of that element.

It should be understood that where the specification states that a component, feature, structure, or characteristic “may”, “might”, “can” or “could” be included, that particular component, feature, structure, or characteristic is not required to be included.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

The terms “bottom”, “below”, “top” and “above” and the like that may be specified herein do not necessarily indicate that a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both such as when viewing them in the figures.

It should be understood that, unless otherwise indicated, the term “couple”, “coupled”, “coupling” and grammatical variations thereof as used herein, refers to the mechanical coupling between a plurality of elements, wherein the mechanical coupling between the plurality of elements may refer to an embodiment in which the different elements are substantially fixedly coupled to each other, as well as to another embodiment in which the plurality of elements may be integrally formed with each other.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

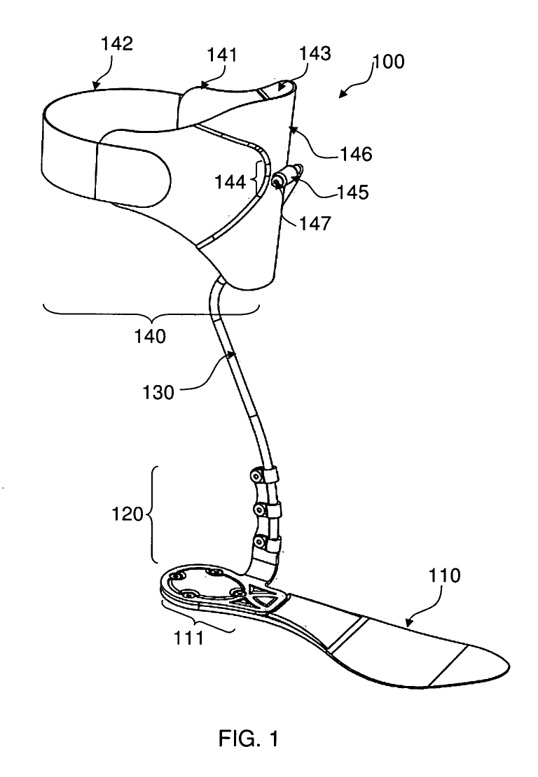

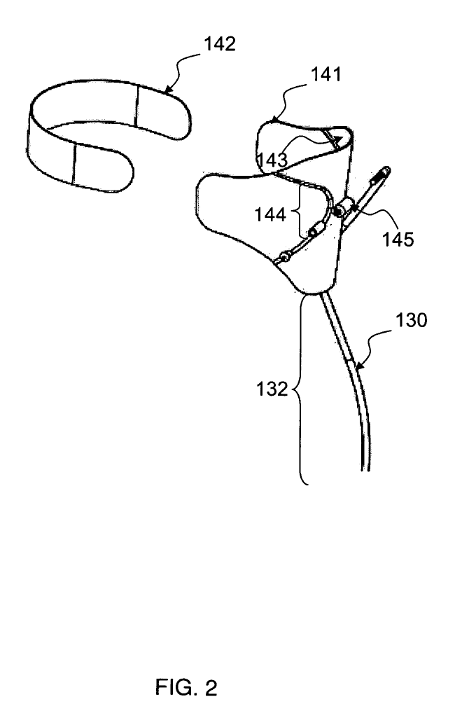

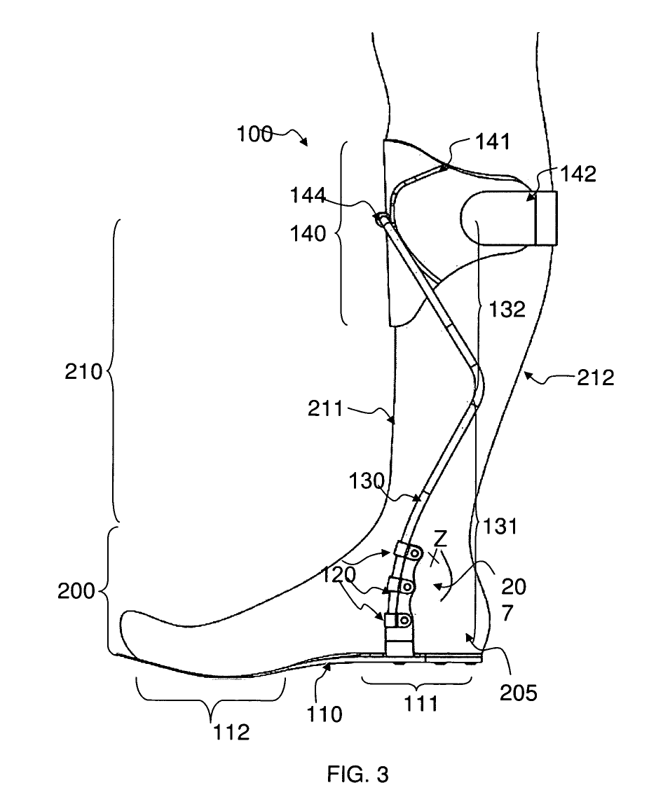

Reference is now made to FIG. 1, FIG. 2 and FIG. 3. According to some embodiments of the invention, an AFO device 100 is designed to provide the patient's drop foot 200 with enough stability so as to enable the patient to walk nearly or substantially regularly. Accordingly, AFO device 100 assists the patient having drop foot 200 to complete substantially regularly a plurality of gait cycles.

According to some embodiments of the invention, AFO device 100 includes an ergonomically designed foot-support 110 that may be adapted, e.g., sized to fit easily, snugly and securely inside a patient's footwear (not shown). Such as footwear may be, for example, a shoe, a sandal, and a boot. In addition, the AFO device 100 is designed to enable comfortable pulling out of the patient's foot from the patient's footwear. Furthermore, the ergonomics and/or the mechanical properties of foot-support 110 may be designed and/or engineered, respectively to enable substantial fitting of foot-support 110 with the natural contour of a sole of a patient's drop foot 200 during all phases of the patient's gait cycle as well as during stance.

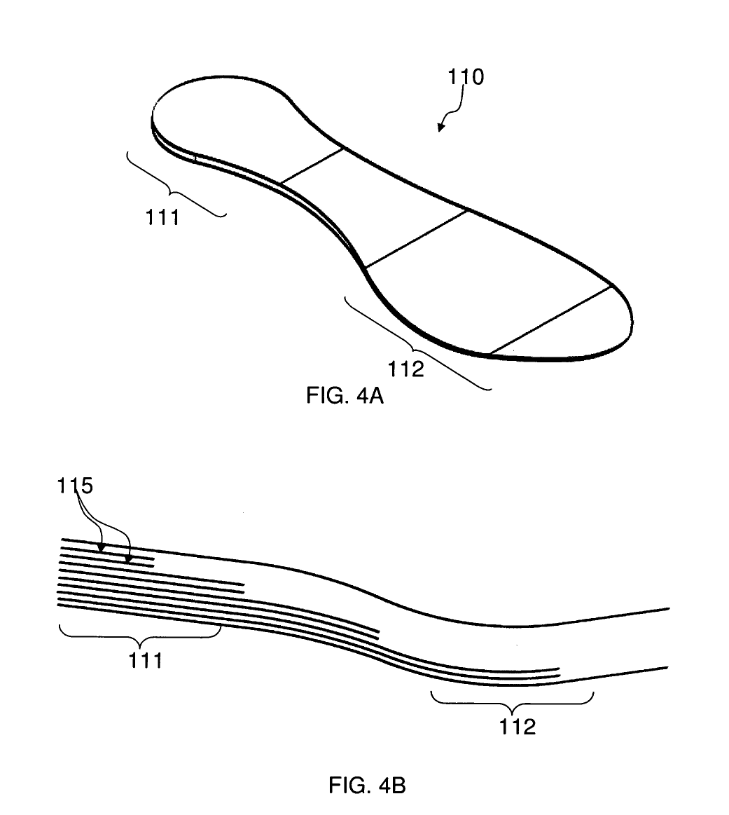

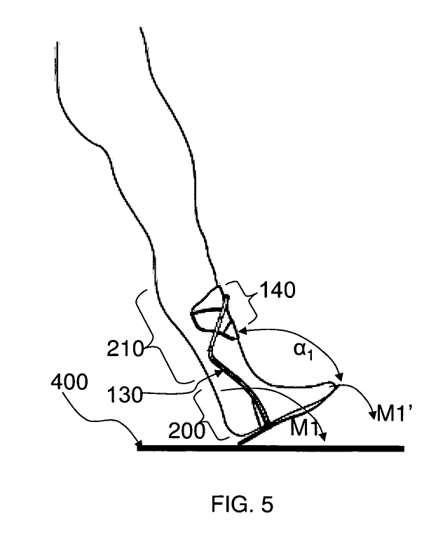

Referring now to FIGS. 4A and 4B, the mechanical properties and/or the ergonomics of foot-support 110 may be engineered, for example, by suitably structuring the material(s) out of which the foot-support 110 is made of, such that foot-support 110 assists in compensating the patient's drop foot condition, which may be neurological and/or muscular and/or anatomic in origin. The material(s) out of which foot-support 110 is made of may be lightweight and strong, as well as flexible. Foot-support 110 may weight, for example, less than 2 kilograms.

In one embodiment, foot-support 110 includes a heel portion 111 and a ball portion 112. Foot-support 110 may be made out of a composite material, e.g., as known in the art, or of any other suitable material. For example, foot-support 110 may comprise of a plurality of layers of a first material disposed within a second material, the first and the second material making up a composite material, e.g., as known in the art. For example, the first material may comprise a plurality of layers of fiber (e.g., carbon fibers, graphite fibers). The fiber layers may be embedded within the second material that may comprise of a thermoset such as, for example, epoxy resin, polydicyclopentadiene, polyimide or any other suitable material. According to some embodiments of the invention, the second material may be disposed relative to each other as to form a matrix-like structure, e.g., as known in the art, wherein the second material may serve as a bond material holding the first material together. Various parameters of the first and/or the second material such as, for example, orientation, thickness, density or other parameters may influence or dictate the mechanical properties of the composite material. For example, the higher the density of the fibers within a layer or a certain portion of the layer, the higher may be the rigidity of the layer or the said portion of the layer. This effect is, for exemplary purposes only, schematically illustrated in FIG. 4B, wherein heel portion 111 of foot-support 110 includes more layers of fibers 115 than ball portion 112. Consequently, heel portion 111 may be less flexible to bending than ball portion 112, as is schematically illustrated by the more extensive bending of ball portion 112 compared to the bending of heel portion 111.

According to some embodiments of the invention, foot-support 110 may be made of additional or alternative materials such as, for example; fiber reinforced polymers (e.g., carbon-fiber reinforced plastic, glass-fiber reinforced plastic); thermoplastic composite material; thermoplastic composites or any other suitable material(s).

Reverting to FIG. 1, FIG. 2 and FIG. 3, in order to make usage of the AFO device 100, the patient places foot-support 110 inside the footwear (not shown) that corresponds to the body side of the patient's drop foot 200, i.e., if the drop foot 200 is the patient's left foot, the patient adjusts foot-support 110 to a left-sided footwear. The patient may then insert his/her drop foot 200 into the corresponding footwear which he/she may then don as usual. Once the foot-support 110 and the drop foot 200 are in place, the patient may secure the AFO device 100 to his/her lower leg 210 by means of a lower-leg-holder 140, as will be described hereinbelow.

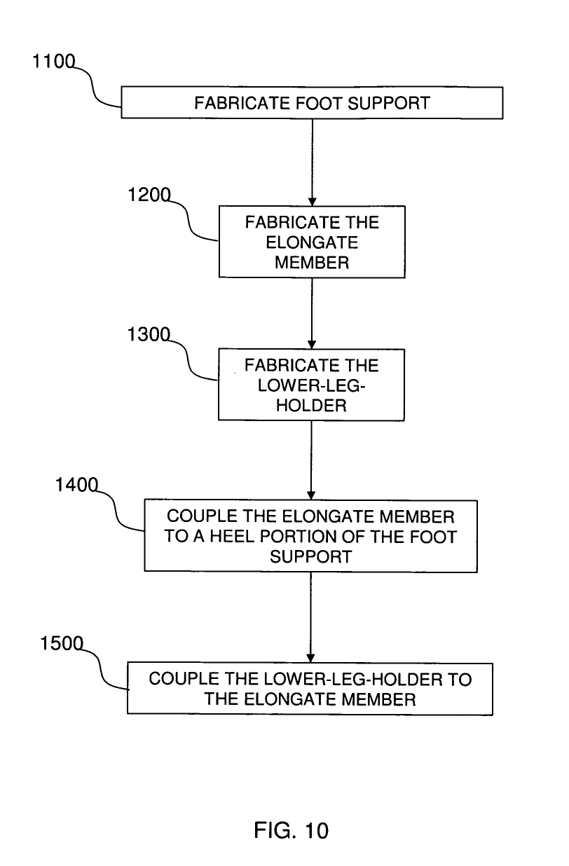

According to some embodiments of the invention, an elongate member 130 is substantially fixedly coupled to heel portion 111 of the foot-support 110 by means of at least one elongate-member-fastener 120, which may be substantially rigid. A number of elongate-member-fastener(s) 120 are described hereinbelow, with respect to FIG. 9. According to some other embodiments of the invention, the elongate member 130 may be integrally formed with the heel portion 111 of the foot-support 110. Elongate member 130 has spring-like properties, i.e., elongate member 130 is flexible.

Elongate member 130 may be bent and may comprise of a lower portion 131 prolonging to an upper portion 132, wherein lower portion 131 may extend rearwardly from heel portion 111 towards calf 212 and wherein upper portion 132 may extend anteriorly towards a corresponding shin 211. Accordingly, the lower portion may be bent relative to the upper portion.

Furthermore, according to some embodiments of the invention, at least some parts of elongate member may be shaped and fixedly coupled to foot-support 110 via elongate-member-fastener(s) 120 such that elongate member 130 substantially intersects the anatomic axis of ankle 207, which is schematically indicated with arrow “Z”. More specifically, lower portion 131 may extend from heel portion 111 substantially in alignment with ankle 207 towards a posterior position with regard to heel bone 205 and ankle 207, wherein the posterior position of lower portion 131 may be substantially above heel bone 205 and ankle 207. Consequently, AFO device 100 is designed and adjustable to drop foot 100 as to enable substantial imitation of the biomechanics of a healthy foot. Due to the alignment of elongate member 130 with anatomic axis Z, AFO device 100 does not interfere with the anatomic axis Z.

According to some embodiments of the invention, the elongate member 130 may be made out of, for example, a resilient steel material such as, for example, high carbon steel, very high carbon steel or any other suitable material.

According to some embodiments of the invention, lower-leg-holder 140 may include a U-shaped support-brace 141 that may be mechanically coupled, e.g., via its outer front part 146, to the end part of the elongate member 130 by leg-holder-fastener(s) 144 such that a curved part of U-shaped support-brace 141 substantially points in a direction that corresponding to the patient's gait. Leg-holder-fastener(s) 144 may be, for example, a sleeve 145 that is fixedly coupled to outer front part 146 and into which said end part is tightly fitted. According to some embodiments of the invention, leg-holder-fastener(s) 144 may further include a securing means 147 such as, for example, a rivet, for preventing the sliding out of said end part from said sleeve 145.

According to some embodiments of the invention, support-brace 141 may be shaped to substantially fit onto at least some part of shin 211, such that lower leg 210 can rest against an inner surface 143 of U-shaped support-brace 141. Furthermore, according to some embodiments of the invention, the AFO device 100 is secured to the patient's lower leg 210 by, e.g., straps 142.

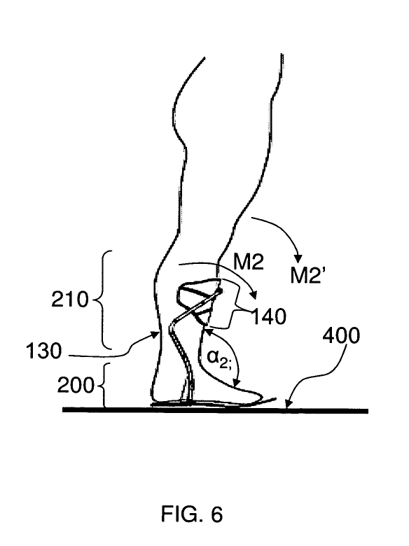

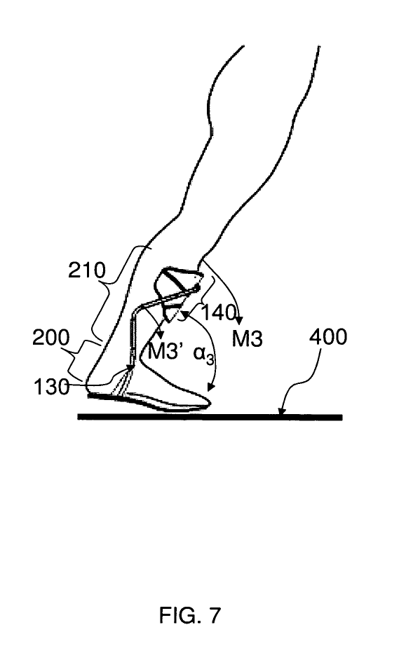

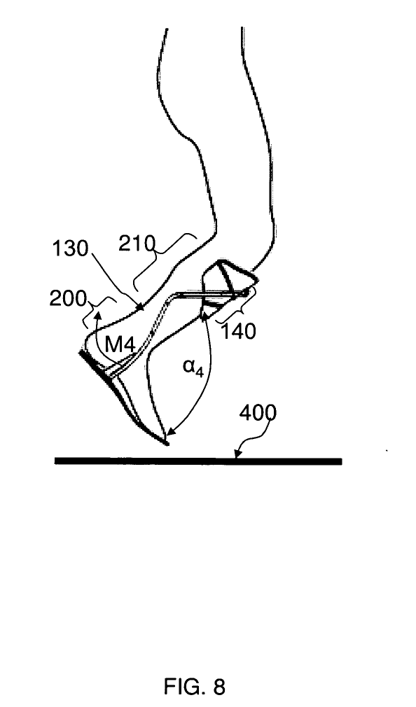

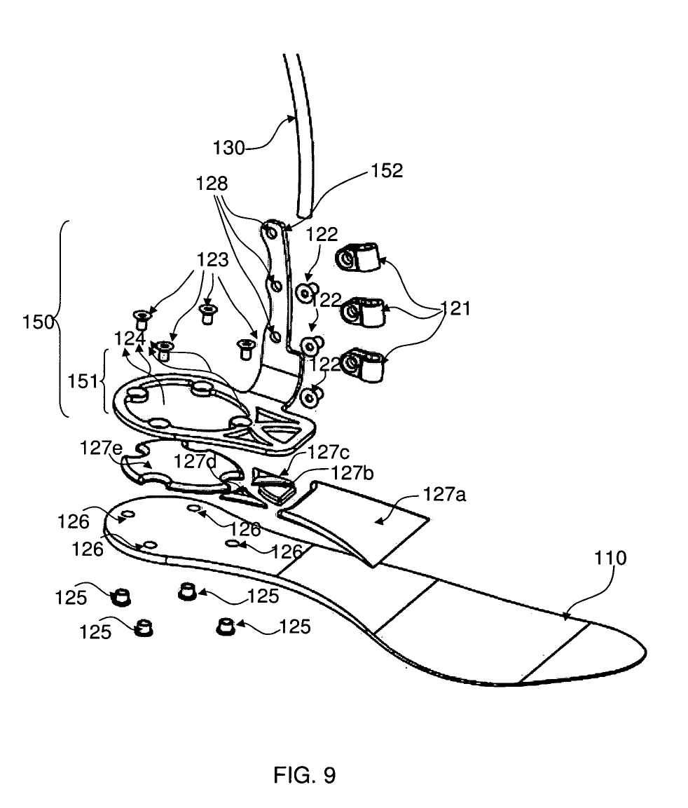

Further reference is now made to FIG. 5, FIG. 6, FIG. 7 and FIG. 8. The following is a description of how AFO device 100 may assist the patient in overcoming the effects of the drop foot condition such that the patient may be able to walk nearly regularly.

It should be understood that the angles α₁, α₂, α₃, α₄, as specified herein are not to be referred to as being angles of fixed size but rather as angles during the initial-contact, mid-stance, terminal-stance and toe-off phase of the gait cycle, respectively, wherein each of the angles may dynamically change during each of the phases.

During the initial-contact phase (FIG. 5), at least some of heel portion 111 of foot-support 110 engages with floor 400. The engagement of heel portion 111 with floor 400 causes drop foot 200 to apply a moment M1 on foot-support 110 around elongate-member-fastener(s) 120 such that the tip of drop foot 200 may pivot away from lower leg 210, as schematically indicated with arrow M1′. During the initial-contact phase, the angle α₁ between the tip of drop foot 200 and shin 211 may be slightly larger than approximately 90°. For example, during initial-contact phase, angle α₁ may have values of for example, approximately 93°, 95° and 97°. Angle α₁ may also have other values during the initial-contact phase.

Upon completion of the initial-contact phase (FIG. 5), the patient may perform a transition to the mid-stance phase (FIG. 6), wherein in the mid-stance phase, both heel portion 111 and ball portion 112 are engaged with floor 400. During the mid-stance phase, lower leg 210 may rest against lower-leg-holder 140, thereby applying a moment M2 against inner surface 143 of support-brace 141 of lower-leg-holder 140. As a result thereof, lower leg 210 may move towards drop foot 200, as schematically indicated with arrow M2′, and the angle α₂ between the tip of foot 200 and shin 211 decreases with respect to angle α₁ to by, for example, approximately 2°-5°, such that α₂ may attain a value of, for example, approximately 90° or approximately 88°. Angle α₂ may also have other values during the mid-stance phase.

Upon completion of the mid-stance phase (FIG. 6), the patient may perform a transition to the terminal-stance phase (FIG. 7), wherein in the terminal-stance phase, heel portion 111 may disengage from floor 400 due to the force and/or moment applied by lower leg 210 against inner surface 143 of U-shaped support-brace 141. During the terminal-stance phase (FIG. 7), the angle α₃ between the tip of foot 200 and shin 211 may further decrease with respect to angle α₂ up to, for example, approximately 85°. Angle α₃ may also have other values during the terminal-stance phase (FIG. 7).

It should be understood that AFO device 100 is adjustable to meet individual patient's needs. For example, AFO device 100 is individually adjustable to a patient's anatomy and biomechanical behavior. Accordingly, AFO device 100 may be adjusted such that during the terminal stance phase (FIG. 7) of a first patient, angle α₃ may decrease up to approximately 83°, whereas during the terminal stance phase of a second patient, angle α₃ may decrease only up to approximately 85°.

In order to perform a transition from the mid-stance phase (FIG. 6) to the terminal-stance phase (FIG. 7), the patient may shift his/her body weight to the gait's direction and towards the side of drop foot 200, whilst tilting lower leg 210 downwards and forwards against the inner surface 143 of support-brace 141 of lower-leg-holder 140. Shifting his/her body weight forward in a direction that corresponds to the patient's gait, may result in a moment M3 on the inner surface 143 of support-brace 141. Since elongate member 130 has some elasticity, moment M3 causes elongate member 130 to flex in a direction that is schematically indicated with arrow M3′. Concurrent to the flexing of elongate member 130, drop foot 200 may pivot towards lower leg 210 to take up for the shortening of the distance between the extremities of elongate member 130 occurring due to the bending thereof.

In consequence, elongate member 130 develops therein some potential energy of which some may be released during the subsequent toe-off phase (FIG. 8) in form of, e.g., moment M4 or other moments and/or forces. During the toe-off phase, both heel portion 111 and ball portion 112 disengage from floor 400. The lower part of elongate member 130 may exert, for example, the moment M4 on the heel portion 111 of foot-support 110. Thusly, moment M4 and/or other forces and/or moments may help compensating for the force lost due to, e.g., muscle weakness in drop foot 200 and thereof assists the patient in performing the toe-off phase without dragging drop foot 200 along floor 400. Correspondingly, AFO device 100 may cause ground clearance of the patient's drop foot 200 during the toe-off phase and during at least some of the subsequent swing phases.

Further reference is now made to FIG. 9. According to some embodiments of the invention, elongate-member-fastener(s) 120 may include a connector 150 that may include a base 151 that may be integrally formed with a protruding plate 152. Plate 152 may be positioned adjacent to the inner side of drop foot 200. According to some embodiments of the invention, plate 152 may be adapted to fixedly couple thereto elongate member 130 by means of, e.g., clamps 121, which may clamp elongate member 130 therein. According to some embodiments of the invention, elongate member 130 may have holes 128 suitable for receiving clamp securing fasteners 122. Clamp securing fasteners 122 may be used for securing clamps 121 onto elongate member 130. Securing fasteners 122 may be, for example, bolts, nuts, rivets, and/or any other suitable fasteners.

According to some embodiments of the invention, base 151 may be fixedly coupled to heel portion 111 of foot-support 110 via, e.g., heel fasteners 123, which may be, for example, bolts, nuts, rivets and the like. According to some embodiments of the invention, base 151 and heel portion 111 may be able to receive heel fasteners 123 via holes 124 and 126, respectively. The position of heel fasteners 123 may be secured by securing means 125, which may be, for example, suitable stoppers.

In some embodiments of the invention, padding elements 127a, 127b, 127c, 127d and 127e may be fitted onto foot-support 110 in a manner that substantially complements the shape of base 151, thereby providing comfortable cushioning to the sole of drop foot 200.

According to some embodiments of the invention, all of the abovementioned elements of AFO device 100 may be replaceable.

In embodiments of the invention, foot-support 110 may approximately weigh, for example, 70 grams, 60 grams, 50 grams, 40 grams or 30 grams.

In embodiments of the invention, elongate member 130 may approximately weigh, for example, 100 grams, 90 grams, 80 grams, 70 grams or 60 grams.

In embodiments of the invention, lower leg holder 140 may approximately weigh, for example, 20 grams, 25 grams, 30 grams, 35 grams, 40 grams, 45 grams or 50 grams.

In embodiments of the invention, AFO device 100 may approximately weigh, for example, 500 grams, 400 grams, 350 grams and the like.

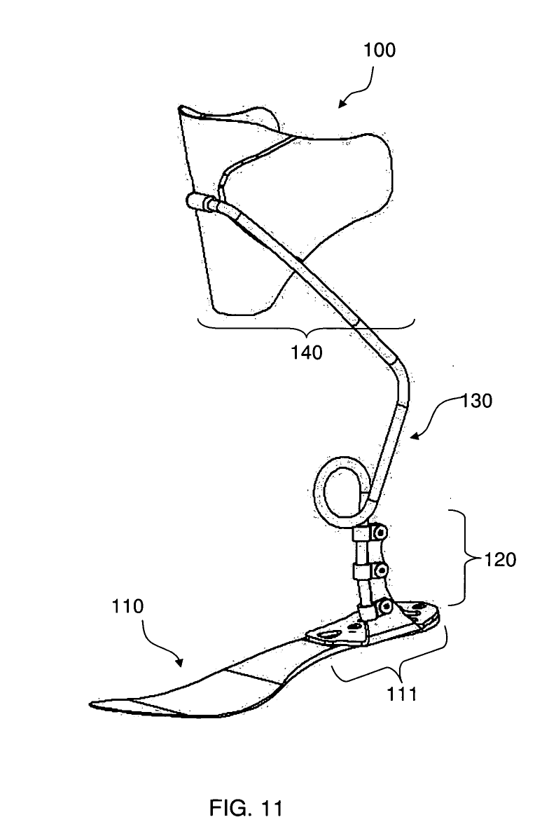

Reference is now made to FIG. 10. According to some embodiments of the invention, as indicated by box 1100, a method for fabricating an ankle foot orthosis device, may include, for example, the act of fabricating foot support 110 by, for example, laying up several layers of, e.g., composite material.

According to some embodiments of the invention, as indicated by box 1200, the method may include, for example, the act of fabricating elongate member 130.

According to some embodiments of the invention, as indicated by box 1300, the method may include, for example, the act of fabricating lower-leg-holder 140.

According to some embodiments of the invention, as indicated by box 1400, the method may include, for example, the act of mechanically coupling elongate member 130 to heel portion 111 of foot-support 110. This may be accomplished in a manner such that lower portion 131 extends rearwardly from heel portion 111 towards drop foot's 200 corresponding calf 212 and such that upper portion 132 extends anteriorly towards the drop foot's 200 corresponding shin 211.

According to some embodiments of the invention, as indicated by box 1500, the method may include, for example, the act of mechanically coupling lower-leg-holder 140 to elongate member 130. This may be accomplished in a manner such that such that the curved part of support-brace 141 substantially points in a direction that corresponds to the patient's direction of gait, thereby enabling the patient to rest his/her lower leg 210 that corresponds to the drop foot 200 against said inner surface 143 of support-brace 141.

While the invention has been described with respect to a limited number of embodiments, these should not be construed as limitations on the scope of the invention, but rather as exemplifications of some of the embodiments. Those skilled in the art will envision other possible variations, modifications, and programs that are also within the scope of the invention. Accordingly, the scope of the invention should not be limited by what has thus far been described, but by the appended claims and their legal equivalents. Therefore, it is to be understood that alternatives, modifications, and variations of the present invention are to be construed as being within the scope and spirit of the appended claims.

What is claimed is:

1. An ankle foot orthosis device adjustable to patient's drop foot for assisting said patient in completing substantially regularly a gait cycle, the device comprising:
a foot-support that is sized to fit into corresponding footwear of said patient's drop foot, said foot support comprising a heel portion;
an elongate member that is mechanically coupled to said foot-support, said elongate member comprising of a lower portion prolonging to an upper portion; and
a lower-leg-holder that is coupled to said elongate member, said lower-leg-holder comprising a support-brace that enables a corresponding shin to rest thereon;
wherein said lower portion of said elongate member extends from said heel portion of said foot-support upwards and rearwards towards a corresponding lower leg's calf of said patient, wherein said upper portion further extends forwardly in a direction corresponding to said lower leg's shin; and
wherein forward tilting of said lower leg against said support-brace causes said elongate member to bend forward in a direction that corresponds to said patient's gait, wherein said forward bending causes the development of potential energy in said ankle foot orthosis device, wherein at least some of said potential energy is released during the toe-off phase of said gait cycle, wherein said potential energy compensates at least partially for the energy lost due to the drop foot condition, thereby causing ground clearance to said drop foot during at least some of the gait cycles swing phases.

2. The ankle foot orthosis device of claim 1, wherein said elongate member is flexible.

3. The ankle foot orthosis device of claim 1, wherein said foot-support is flexible.

4. The ankle foot orthosis device of claim 1, wherein said foot-support weighs less than approximately 2 kilograms.

5. The ankle foot orthosis device of claim 1, wherein said foot-support comprises of at least one of the following first materials: carbon fibers and graphite fibers.

6. The ankle foot orthosis device of claim 15, wherein said first material is disposed within at least one of the following second materials: Epoxy Resin, Polydicyclopentadiene and Polyimide.

7. The ankle foot orthosis device of claim 5, wherein the density and the structure of said first material dictates mechanical properties of said foot-support.

8. The ankle foot orthosis device of claim 6, wherein the density and the structure of said second material dictates mechanical properties of said foot-support.

9. The ankle foot orthosis device of claim 6, wherein the density and the structure of said second material dictates mechanical properties of said foot-support.

10. The ankle foot orthosis device of claim 1, wherein said foot-support is ergonomically designed to substantially fit the natural contour of a sole of the drop foot of said patient.

11. The ankle foot orthosis device of claim 1, wherein said foot-support is sized to fit easily, snugly and securely inside said patient's corresponding footwear.

12. The ankle foot orthosis device of claim 8, wherein said mechanical properties of said foot-support are engineered as to enable substantial fitting of said foot-support with the natural contour of a sole of a patient's drop foot during all phases of the patient's gait cycle.

13. The ankle foot orthosis device of claim 1, wherein said elongate member is integrally formed with said foot-support.

14. The ankle foot orthosis device of claim 1, wherein said elongate member is fixedly coupled to said foot-support with at least one elongate-member-fastener.

15. The ankle foot orthosis device of claim 1, wherein said support-brace is secured to some portion of said shin by straps.

16. The ankle foot orthosis device of claim 1, wherein said elongate member is made of at least one of the following materials: high carbon steel, and very high carbon steel.

17. The ankle foot orthosis device of claim 1, wherein at least some parts of said elongate member is shaped and fixedly coupled to foot-support via said at least one elongate-member-fastener such that elongate member is substantially aligned with the anatomic axis of the ankle of the patient's drop foot.

18. The ankle foot orthosis device of claim 1, wherein said lower portion of said elongate member extends from a heel portion substantially in alignment with the drop foot's ankle in a direction corresponding to a posterior position with regard to the drop foot's heel bone and ankle, wherein said posterior position is substantially above said heel bone and said ankle.

19. The ankle foot orthosis device of claim 1, wherein at least one of the following elements is replaceable: said at least one elongate-member-fastener, said elongate member and said foot-support.

20. The ankle foot orthosis device of claim 1, wherein said at least one elongate-member-fastener includes a connector comprising of a base that is integrally formed with a protruding plate, wherein said protruding plate is positioned adjacent to the inner side of drop foot, said protruding plate enabling the fixedly coupling of said elongate member to said protruding plate by fastener means.

21. The ankle foot orthosis device of claim 1, wherein padding elements are fitted onto said foot-support to provide comfortable cushioning for said drop foot's sole.

22. The ankle foot orthosis device of claim 1, wherein said foot-support has approximately one of the following weights: 70 grams, 60 grams, 50 grams, 40 grams and 30 grams.

23. The ankle foot orthosis device of claim 1, wherein said elongate member has approximately one of the following weights: 100 grams, 90 grams, 80 grams, 70 grams and 60 grams.

24. The ankle foot orthosis device of claim 1, wherein said lower-leg-holder has approximately one of the following weights: 20 grams, 25 grams, 30 grams, 35 grams, 40 grams, 45 grams and 50 grams.

25. The ankle foot orthosis device of claim 1, wherein said ankle foot orthosis device has approximately one of the following weighs: 500 grams, 400 grams and 350 grams.

26. A method of fabricating an ankle foot orthosis device for assisting a patient having a drop foot, said method comprising:
the act of fabricating a foot support comprising of a heel portion;
the act of fabricating an elongate member comprising of a lower and an upper portion that are bent relative to each other;
the act of fabricating a lower-leg-holder, said lower leg holder comprising of a U-shaped support-brace having an inner surface;
the act of mechanically coupling said elongate member to a heel portion of said foot-support such that said lower portion of said elongate member extends rearwardly from said heel portion towards said drop foot's corresponding calf and such that said upper portion extends anteriorly towards said drop foot's corresponding shin; and
the act of mechanically coupling said lower-leg-holder to said elongate member such that a curved part of said support-brace substantially points in a direction that corresponds to said patient's direction of gait, thereby enabling said patient to rest his/her lower leg that corresponds to said drop foot against said inner surface of said support-brace.
